(19) United States
(12) Patent Application Publication
(10) Pub. No.: US 2006/0189995 A1
(43) Pub. Date: Aug. 24, 2006

(54) Rongeur that disassembles for cleaning

(75) Inventor: Mike Lancial, St. Louis Park, MN (US)

Correspondence Address: FAEGRE & BENSON LLP;PATENT DOCKETING, 2200 WELLS FARGO CENTER, 90 SOUTH 7TH STREET, MINNEAPOLIS, MN, 55402-3901, US

(73) Assignee: Zimmer Spine, Inc., Minneapolis, MN (US)

(21) Appl. No.: 11/063,702

(22) Filed: Feb. 22, 2005

(51) Int. Cl. A61B 17/32

(52) U.S. Cl. 606/83

(57) ABSTRACT

An embodiment of a rongeur includes an assembly pin that provides easy disassembly and assembly of the rongeur. The assembly pin also serves the purpose of coupling a driving member to a top shaft of the rongeur. The assembly pin can be engaged with the driving member for normal operation of the rongeur. Alternatively, the assembly pin can be disengaged from the driving member to decouple the driving member from the top shaft, thereby enabling removal of the top shaft for cleaning. An embodiment of a process for disassembling a rongeur includes pressing an assembly pin engaged with a hook portion of the driver member, said pressing forcing a plug portion of the assembly pin out of the aperture in the top shaft to disengage the assembly pin from the hook portion of the driver member. The process may further include decoupling the top portion from the driver member by passing a neck portion of the assembly pin through a passage defined by the hook portion of the driver member. The process may further include removing the top shaft from the rongeur.

### BACKGROUND

A rongeur is an instrument for removing small rough portions of bone during surgery. After use during a surgical operation, the rongeur is typically sterilized and can be used again. Although sterilization generally cleans the rongeur sufficiently for most uses, it has been found that various debris, such as tissue, can become compacted into various parts of the rongeur, such that the sterilization process does not effectively remove the debris. Such debris can create unsanitary conditions. Over time, after multiple surgeries, the rongeur can become filled with the debris, to the extent that the debris hinders operation of the rongeur.

For example, in punches (a type of rongeur), a top shaft slides along a bottom shaft to pinch bone between the end of the top shaft and a raised distal tip on the bottom shaft. In such rongeurs, the top shaft and bottom shaft are typically coupled tightly. For example, in some implementations, the bottom shaft has a T-slot in which a T-shaped member from the top shaft glides during the forward and backward motion of the top shaft. In traditional punches, the top shaft is not readily removable from the bottom shaft. When using the punch during surgery to punch unwanted bone fragments, debris can get lodged in the T-slot and between the top shaft and the bottom shaft. Traditional sterilization approaches typically do not remove such debris. In addition, because the top shaft is not easily removed from the bottom shaft, such debris is typically not removed, but remains lodged in the punch, where it can create unsanitary conditions and can impede the motion of the top shaft during subsequent uses in surgical operations.

Thus, a need exists for a rongeur that can be disassembled for cleaning.

### SUMMARY

Embodiments of rongeurs described herein include an assembly pin that provides easy disassembly and assembly of the rongeur. The assembly pin also serves the purpose of coupling a driving member to a top shaft of the rongeur. The assembly pin can be engaged with the driving member for normal operation of the rongeur. Alternatively, the assembly pin can be disengaged from the driving member to decouple the driving member from the top shaft, thereby enabling removal of the top shaft for cleaning.

Embodiments of rongeurs described herein include an assembly pin that provides easy disassembly and assembly of the rongeur. The assembly pin also serves the purpose of coupling a driving member to a top shaft of the rongeur. The assembly pin can be engaged with the driving member for normal operation of the rongeur. Alternatively, the assembly pin can be disengaged from the driving member to decouple the driving member from the top shaft, thereby enabling removal of the top shaft for cleaning.

A particular embodiment of a rongeur includes a bottom shaft having a distal tip, a top shaft slideably coupled to a top surface of the bottom shaft, a laterally moveable assembly pin extending into a cavity in the top shaft, the assembly pin including a neck portion disposed between a head portion and a plug portion. The plug portion is wider than the neck portion, and the head portion has an exposed surface enabling pressing of the assembly pin. A driver member extends through the bottom shaft and into the top shaft, coupling to the top shaft by way of the assembly pin. A portion of the assembly pin extends into a slot formed by a hook portion of the driver member. The hook portion defines a channel having a width that allows the neck portion to pass therethrough, but does not allow the plug portion to pass therethrough. When the exposed surface of the head portion is pressed, the assembly pin moves laterally within the cavity, thereby disengaging the driver member from the assembly pin.

An embodiment of a process for disassembling a rongeur includes pressing an assembly pin engaged with a hook portion of the driver member, said pressing forcing a plug portion of the assembly pin out of the aperture in the top shaft to disengage the assembly pin from the hook portion of the driver member. The process may further include decoupling the top portion from the driver member by passing a neck portion of the assembly pin through a passage defined by the hook portion of the driver member. The process may further include removing the top shaft from the rongeur.

Another embodiment of a rongeur includes a bottom shaft having an axially aligned groove and a top shaft having a coupling member fitting into the groove and enabling the top shaft to slide forward and backward on top of the bottom shaft. A driver member is coupled to the top shaft to actuate forward and backward movement of the top shaft. Means for coupling the driver member to the top shaft engage the driver member with the top shaft to cause the forward and backward movement. The means are further configured to enable decoupling of the top shaft from the driver member and removal of the top shaft from the rongeur.

A more complete understanding of the present invention may be derived by referring to the detailed description of preferred embodiments and claims when considered in connection with the figures.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates an exemplary rongeur that can be disassembled for cleaning in accordance with one embodiment of the invention;

FIG. 2 illustrates a portion of a rongeur including an laterally moveable assembly pin having an exposed surface that can be pressed in to disengage the driver member from the assembly pin and remove the top shaft from the rongeur in accordance with one embodiment of the invention;

FIGS. 3A-3B illustrates cross-sections of a portion of a rongeur wherein the assembly pin is not pressed and the driver member is engaged with the assembly pin in accordance with one embodiment of the invention;

FIGS. 4A-4B illustrates other cross-sections of a portion of a rongeur wherein the assembly pin is pressed and the driver member is disengaged from the assembly pin in accordance with one embodiment of the invention;

FIG. 5 illustrates a portion of a disassembled rongeur wherein the top shaft has been removed from the rongeur in accordance with one embodiment of the invention;

FIGS. 6-7 illustrate portions of opposite sides of a rongeur including a laterally moveable assembly pin enabling removal of the top shaft in accordance with one embodiment of the invention; and

FIG. 8 illustrates an exemplary embodiment of a process for disassembling a rongeur in accordance with an embodiment of the present invention.

### DETAILED DESCRIPTION

Exemplary embodiments of rongeurs described herein include an assembly pin that provides easy disassembly and assembly of the rongeur. The assembly pin also serves the purpose of coupling a driving member to a top shaft of the rongeur. The assembly pin can be engaged with the driving member for normal operation of the rongeur. Alternatively, the assembly pin can be disengaged from the driving member to decouple the driving member from the top shaft, thereby enabling removal of the top shaft for cleaning.

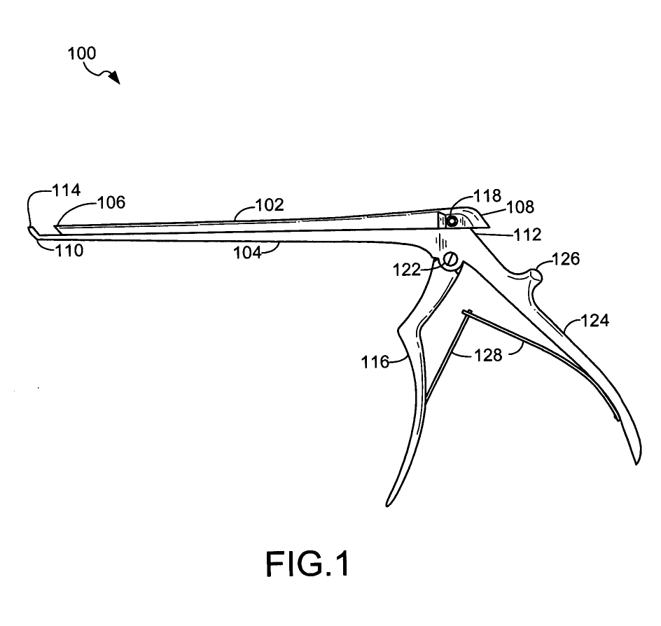

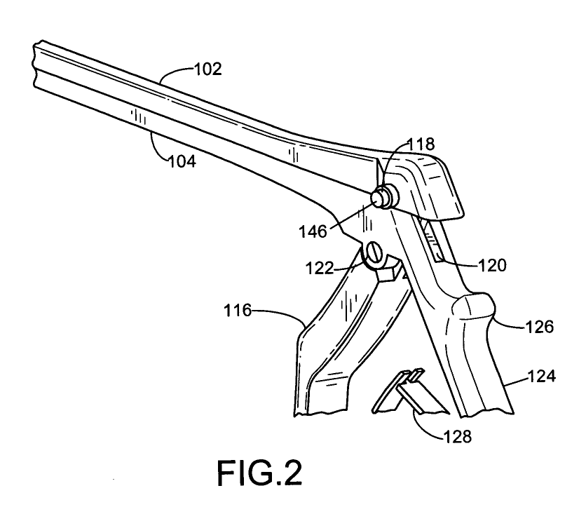

FIG. 1 illustrates an exemplary rongeur 100 that can be disassembled for cleaning in accordance with one embodiment of the invention. FIG. 2 illustrates a portion of the proximal end of the rongeur 100. FIGS. 1-2 are discussed together. The illustrated embodiment of the rongeur 100 includes a top shaft 102 and a bottom shaft 104. The top shaft 102 has a distal end 106 and a proximal end 108. Similarly, the bottom shaft 104 has a distal end 110 and proximal end 112. A distal tip 114 is integrated at the distal end 110 of the bottom shaft 104 to enable pinching of bone or other matter. The top shaft 102 is coupled to the bottom shaft 104 and can slide forward (i.e., toward the distal tip 114) and backward toward the proximal end 112, during operation of the rongeur 100.

In the illustrated embodiment, a driver member 116 is coupled to the top shaft 102 via an assembly pin 118. As discussed in more detail below, the driver member 116 can be disengaged from or engaged with the assembly pin 118. When the driver member 116 is engaged with the assembly pin 118, the driver member 116 is used to apply force to drive the top shaft 102 forward or backward. The force is applied through the driver member 116 to the assembly pin 118, which transfers the forward or backward force to the top shaft 102.

In this particular embodiment, the driver member 116 is a front grip that extends through an aperture 120 (FIG. 2) in the bottom shaft 104. This embodiment of the driver member 116 is pivotally coupled to the bottom member 104 via pivot pin 122. A back grip 124 integrated at the proximal end 112 of the bottom shaft 104 enables a user to squeeze the driver member 116 in a backward direction. The back grip 124 includes a thumb support 126 to facilitate user gripping and squeezing the driver member 116. In this embodiment, the pivot pin 122 leverages the squeezing force into a forward force acting on the assembly pin 118 to move the top shaft 102 forward. Springs 128 mounted between the back grip 124 and the driver member 116 force the driver member 116 back to the resting position when the driver member 116 is released. When the driver member 116 is forced back to the resting position, the assembly pin 118 is pulled backward, which in turn slides the top shaft 102 backward.

In other embodiments, the driver member is not limited to a front grip as shown in FIG. 1. In another embodiment, for example, the driver member can be a trigger, that can be pulled by the user's index finger. In addition, the driver member need not be pivotally coupled to the bottom shaft, such that leverage is applied through a squeezing motion. As an example, in another embodiment, the driver member could be directly coupled (i.e., not by way of pivot pin 122) to the assembly pin, whereby the user pushes the driver member forward in order to actuate forward motion of the top shaft.

After the rongeur 100 has been used to remove bone or other matter, for example, during surgical operations, tissue and other debris can become lodged between the top shaft 102 and the bottom shaft 104. Such debris lodged in the rongeur 100 can have undesirable consequences. For example, such debris can create unsanitary conditions, impede movement of the top shaft 102, and/or result in other undesirable situations. Advantageously, the rongeur 100 includes the assembly pin 118 that can be disengaged from the driver member 116 to enable disassembly of the rongeur 100 by removing the top shaft 102. With the top shaft 102 removed, debris can be more effectively removed from the top shaft 102 and the bottom shaft 104. In addition, in various embodiments, other parts of the rongeur 100, such as the driver member 116, can easily be removed for cleaning by pressing the assembly pin 118.

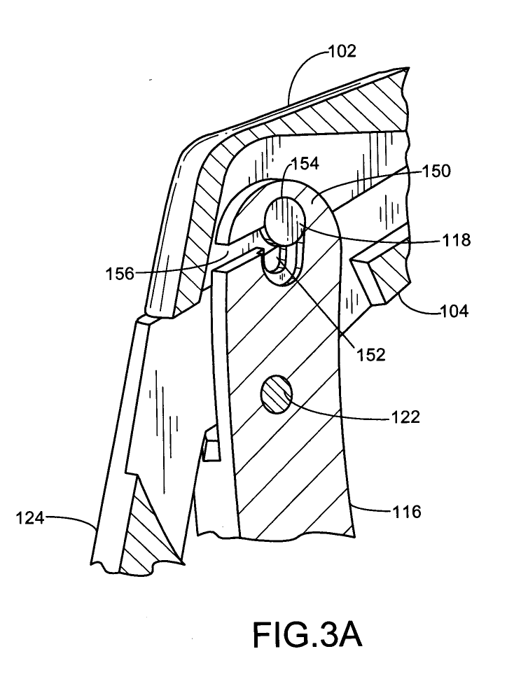

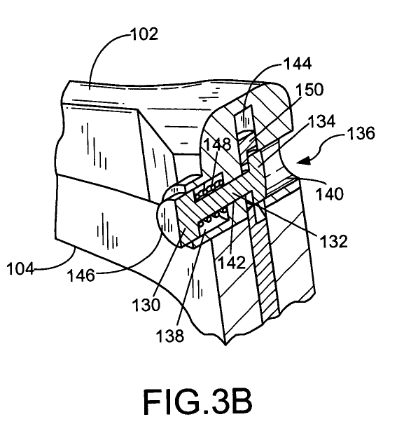

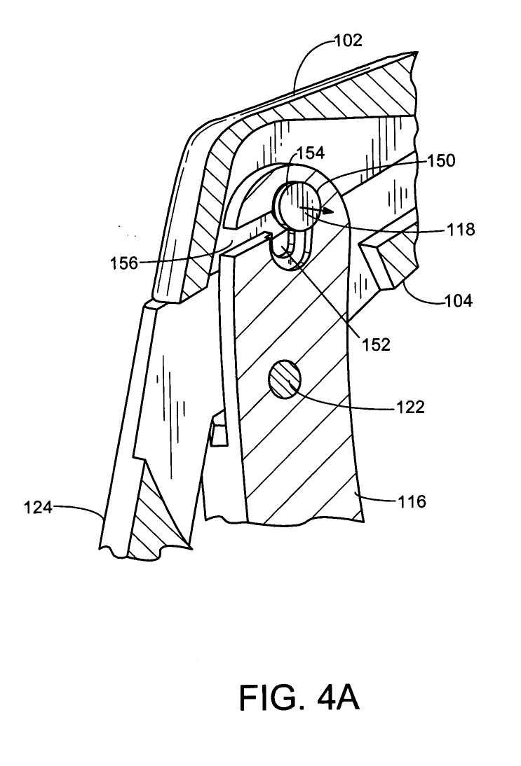

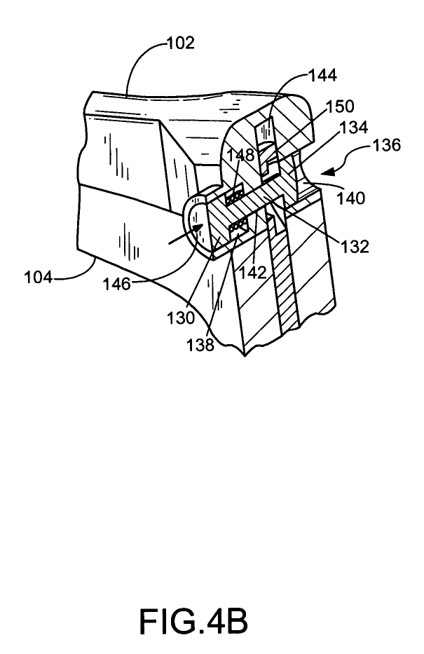

FIGS. 3-4, are now discussed together. FIGS. 3-4 each illustrate cross-sections of a proximal portion of the rongeur 100 depicting one embodiment of the driver member 116 being coupled with the top shaft 102 by way of the assembly pin 118. FIGS. 3A-3B illustrate the assembly pin 118 being engaged with the driver member 116, while FIGS. 4A-4B illustrate the assembly pin 118 being disengaged from the driver member 116.

In the particular embodiment shown in FIGS. 3-4, the assembly pin 118 includes a head portion 130, a neck portion 132, and a plug portion 134. The assembly pin 118 extends into a cavity 136 in the top shaft 102. The cavity 136 is composed of a first chamber 138, a second chamber 140, and a passage 142 between the first chamber 138 and the second chamber 140. An aperture 144 extends perpendicularly through the cavity 136 between the passage 142 and the second chamber 140.

In this particular embodiment, the first chamber 138 is sufficiently wide to fit the head portion 130 therein when a user presses an exposed surface 146 of the head portion 130. The neck portion 132 extends from the head portion 130 through the passage 142 and into the aperture 144. The plug portion 134 is connected to the neck portion 132 and is positioned in the aperture 144 in the engaged position. When the exposed surface 146 of the head portion 130 is pressed, the neck portion 132 slides laterally through the passage 142 and further into the aperture 144, and the plug portion 134 is pushed laterally into the second chamber 140.

In the particular embodiment shown, the first chamber 138 houses a compression spring 148. The compression spring 148 can be coiled around the neck portion 132, such that when the assembly pin 118 is pressed, the spring 148 compresses between an unexposed surface of the head portion 130 and an inner wall of the top shaft 102. FIG. 4B illustrates the assembly pin 118 in the pressed position and the spring in the compressed position.

In the particular embodiment shown, the cavity 136 is open on both sides of the rongeur 100. However, in other embodiments, the cavity 136 can be closed on the side of the second chamber 140 that receives the plug portion 134; i.e., on the side of the top shaft 102 opposite the head portion 130 of the assembly pin 118. In these embodiments, the second chamber 140 can include a spring disposed between the bottom of the plug portion 134 and a wall of the second chamber 140. In this position, the spring can push the assembly pin 118 back into the engaged position when the assembly pin 118 is not being pressed.

In the embodiment shown in FIGS. 3-4, the top of driver member 116 includes an integrated hook portion 1 50 for engaging with the assembly pin 118. The hook portion 150 extends into the aperture 144 in the top shaft 102. The hook portion 150 defines an engagement slot 152 into which the plug portion 134 fits. In this exemplary embodiment, in the engaged position, the hook portion 150 hooks over a shoulder 154 of the plug portion 134 to couple the driver member 116 with the top shaft 102. When the driver member 116 is pulled backward in the engaged position (FIG. 3), the driver member 116 pivots on the pivot pin 122, and the hook portion 150 pulls the assembly pin 118 forward, thereby actuating the top shaft 102 in a forward direction.

In the particular embodiment of FIGS. 3-4, the hook portion 150 of the driver member 116 defines a channel 156. The channel 156 is more narrow than the plug portion 134. Thus, passage of the plug portion 134 is not allowed through the channel 156. However, the channel 156 is wide enough to allow the neck portion 132 to pass therethrough. When the assembly pin 130 is pressed (as shown in FIG. 4), the plug portion 134 moves out of the engagement slot 152 and into the second chamber 140. Also when the assembly pin 118 is pressed, the neck portion 132 is moved farther into the aperture 144. As such, when the assembly pin 118 is pressed, the neck portion 132 is in line with the channel 156. By sliding the neck portion 132 through the channel 156, the driver member 116 is disengaged from the assembly pin 118.

In the exemplary embodiment shown in FIGS. 3-4, the neck portion 132 of the assembly pin 118 can be passed through the channel 156 by moving the hook portion 150 forward (i.e., toward the distal end 106) or by moving the neck portion 132 backward (i.e., toward the proximal end 108). The neck portion 132 may be moved backward through the channel 156 by grasping the top shaft 102 and pulling the top shaft 102 backward while the assembly pin 118 is pressed. The driver member 116 may be moved forward to pass the neck portion 132 through the channel 156 by squeezing the driver 116 and the back grip 124 together while the assembly pin 118 is pressed.

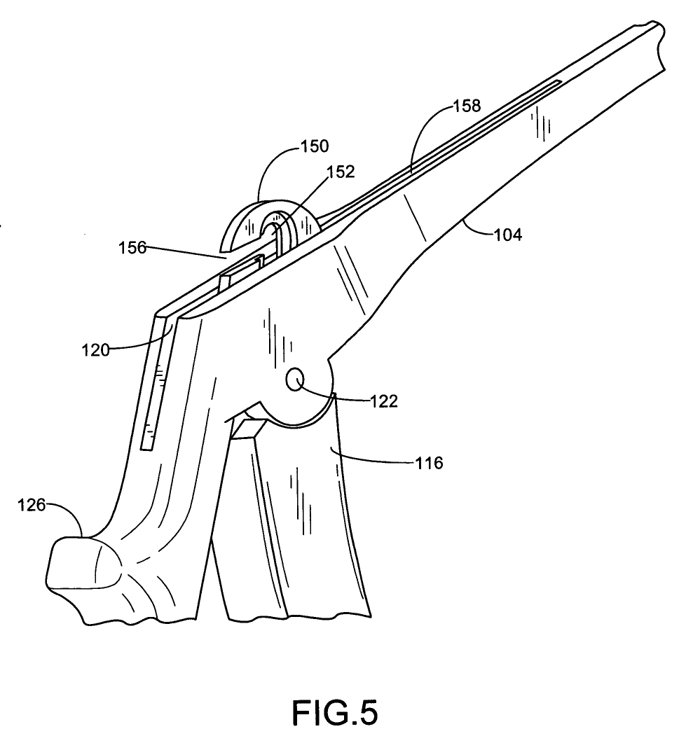

FIG. 5 illustrates a portion of a disassembled rongeur 100 wherein the top shaft 102 has been removed from the rongeur 100 in accordance with one embodiment of the invention. The reader can clearly see the aperture 120 in the bottom shaft 104 through which the driver member 116 passes. When the top shaft 102 is removed as shown, the hook portion 150 of the driver member 116 extends through the bottom shaft 104, with the channel 156 toward the proximal end 112 of the bottom shaft 104.

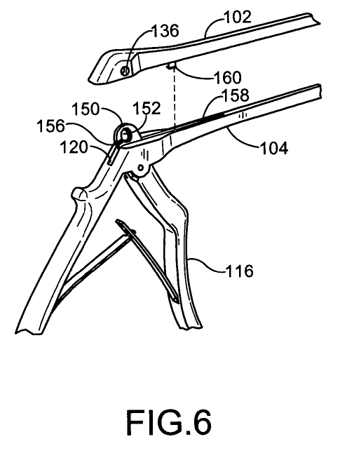

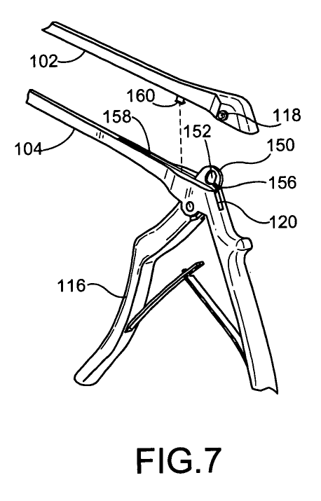

FIGS. 6-7 illustrate portions of opposite sides of a disassembled rongeur 100 including the laterally moveable assembly pin 118 enabling removal of the top shaft 102 in accordance with one embodiment of the invention. In this embodiment, the bottom shaft 104 includes an axially aligned T-shaped groove 158 configured to receive a T-shaped member 160 protruding downward from the top shaft 102. The top shaft 102 can be slideably coupled to the bottom shaft 104 by inserting the T-shaped member 160 into the T-shaped groove 158. The top shaft 102 can be coupled to the driver member 116 by pressing the assembly pin 118 and guiding the neck portion 132 of the assembly pin 118 through the channel 156 of the hook portion 150. Once the rongeur 100 is assembled, the T-shaped groove 158 guides the T-shaped member 160 during forward and backward actuation of the top shaft 102. It will be understood that other embodiments are not limited to a T-shaped groove 158 and T-shaped member 160 for coupling the top member 102 to the bottom member 104.

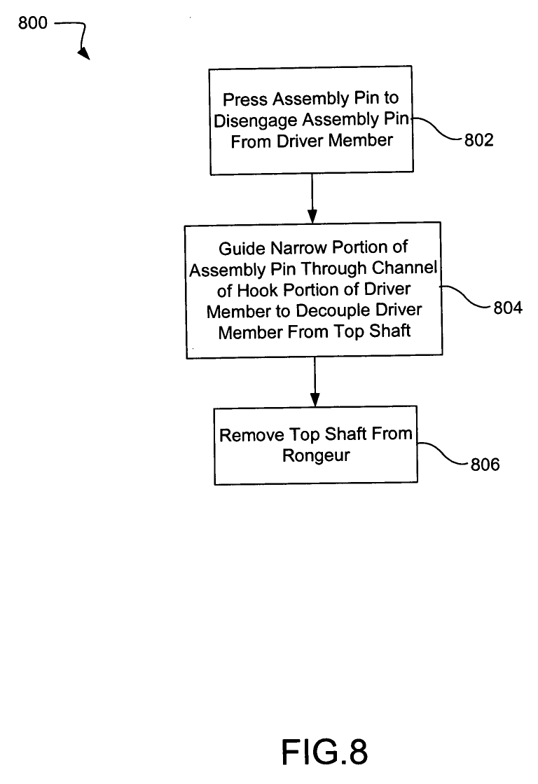

FIG. 8 illustrates an exemplary embodiment of a process 800 for disassembling a rongeur in accordance with an embodiment of the present invention. In a pressing operation 802, the assembly pin is pressed to disengage the assembly pin from the driver member (e.g., a squeezable front grip, trigger, etc.). A guiding operation 804 guides a narrow portion (e.g., neck portion 132) of the assembly pin through a channel in a top portion (e.g., hook portion 150) of the driver member. The narrow portion may be guided through the channel by moving the top shaft backward, moving the driver shaft forward, or a combination of both. Once the narrow portion of the assembly pin has passed through the channel, the driver member is decoupled from the top shaft. A removing operation 806 removes the top shaft from the rongeur, enabling cleaning of the top shaft and other parts of the rongeur.

Various exemplary devices and methods have been illustrated in the accompanying drawing and described in the foregoing detailed description. It will be understood that the methods and devices shown and described are not limited to the particular embodiments described herein, but rather are capable of numerous rearrangements, modifications, and substitutions without departing from the scope and spirit of the claims set forth below.

What is claimed is:

1. A rongeur comprising:
a bottom shaft having a distal tip;
a top shaft slideably coupled to a top surface of the bottom shaft;
a laterally moveable assembly pin extending into a cavity in the top shaft, the assembly pin including a neck portion disposed between a head portion and a plug portion, the plug portion is wider than the neck portion, the head portion having an exposed surface enabling pressing of the assembly pin;
a driver member extending through an aperture in the bottom shaft and into an aperture in the top shaft and coupling to the top shaft by way of the assembly pin,
wherein at least a portion of the assembly pin extends into the aperture and through a slot formed by a hook portion of the driver member, the hook portion defining a channel having a width that allows the neck portion to pass therethrough, but does not allow the plug portion to pass therethrough;
wherein the cavity is composed of a passage having a width sufficient to fit the neck portion, a first chamber having a width sufficient to fit the head portion therein, and a second chamber having a width sufficient to fit the plug portion therein, wherein the passage is between the first chamber and the aperture, and the aperture is between the passage and the second chamber; and
wherein when the exposed surface of the head portion is pressed, the plug portion exits the slot and enters the second chamber, and the neck portion enters the slot, thereby disengaging the driver member from the assembly pin.

2. A rongeur as recited in claim 1 wherein the top shaft can be decoupled from the driver member when the driver member is disengaged from the assembly pin by passing the neck portion through the channel defined by the hook portion.

3. A rongeur as recited in claim 1 further comprising a spring housed in the cavity, the spring abutting a surface of the assembly pin, the spring forcing the plug portion into the slot thereby engaging the assembly pin with the driver member.

4. A rongeur as recited in claim 3 wherein the head portion of the assembly pin is wider than the neck portion, and wherein a spring abuts an unexposed surface of the head portion in the first chamber.

5. A rongeur as recited in claim 3 wherein the second chamber is closed on a side of the top shaft that is opposite the head portion, and the spring abuts an unexposed surface of the plug portion in the second chamber.

6. A rongeur as recited in claim 1 wherein disengagement of the driver member from the assembly pin enables removal of the top shaft from the bottom shaft.

7. A rongeur as recited in claim 1, wherein the driver member is disengaged from the assembly pin by moving the driver member, such that the neck portion passes through the passage defined by the hook portion of the driver member.

8. A rongeur as recited in claim 1, wherein the driver member is disengaged from the assembly pin by sliding the top shaft back, such that the neck portion passes through the passage defined by the hook portion of the driver member.

9. A rongeur as recited in claim 1 further comprising:
a rear grip attached to a proximal end of the bottom shaft;
one or more springs disposed between the driver member and the rear grip enabling squeezing of the driver member toward the rear grip, and when the driver member is engaged with the laterally moveable assembly pin, said squeezing causing the hook portion of the driver member to apply a forward force on the laterally moveable assembly pin, thereby sliding the top shaft along the bottom shaft toward the distal tip.

10. A rongeur as recited in claim 1 wherein the driver member comprises a trigger.

11. A method for cleaning a rongeur having a top shaft slideably coupled to a bottom shaft, a driver member extending through an aperture in the bottom shaft and coupled to the top shaft by way of an assembly pin extending into the top shaft, wherein a force on the driver member causes the top shaft to slide forward or backward along the bottom shaft, the method comprising:
pressing an assembly pin engaged with a hook portion of the driver member, said pressing forcing a plug portion of the assembly pin out of the aperture in the top shaft to disengage the assembly pin from the hook portion of the driver member;
decoupling the top portion from the driver member by passing a neck portion of the assembly pin through a passage defined by the hook portion of the driver member; and
removing the top shaft from the rongeur.

12. A method as recited in claim 11 wherein the driver member comprises a front grip, the rongeur further comprising a back grip and springs disposed between the front grip and the back grip, and wherein decoupling the top shaft from the driver member comprises squeezing the front grip while the pressing the assembly pin, said squeezing causing the neck portion of the assembly pin to pass through the channel defined by the hook portion.

13. A method as recited in claim 11 wherein decoupling the top shaft from the driver member comprises pulling the top shaft back while pressing the assembly pin, said pulling causing the neck portion of the assembly pin to pass through the channel defined by the hook portion.

14. A method as recited in claim 11 wherein the driver member comprises a trigger.

15. A method as recited in claim 11 further comprising removing the driver member from the rongeur.

16. A rongeur comprising:
a bottom shaft having an axially aligned groove;
a top shaft having a coupling member fitting into the groove and enabling the top shaft to slide forward and backward on top of the bottom shaft;
a driver member coupled to the top shaft actuating forward and backward movement of the top shaft; and
means for coupling the driver member to the top shaft, said means engaging the driver member with the top shaft to cause said forward and backward movement, said
means further configured to enable decoupling of the top shaft from the driver member and removal of the top shaft from the rongeur.

17. A rongeur as recited in claim 16, wherein the means for coupling comprises a laterally moveable assembly pin extending into a cavity of the top member and through an engagement slot formed by a top portion of the driver member, the top portion further defining a channel enabling passage of a narrow portion of the assembly pin when the assembly pin is pressed, but disabling passage of a wide portion of the assembly pin when the assembly pin is not pressed.

18. A rongeur as recited in claim 17 wherein the driver member is a squeezable front grip.

19. A rongeur as recited in claim 17 wherein the cavity houses a spring for pushing the assembly pin out of the cavity when the assembly pin is not pressed.

20. A rongeur as recited in claim 17 wherein the top portion of the driver member is substantially hook-shaped.
